(19) United States
(12) Patent Application Publication
(10) Pub. No.: US 2021/0298863 A1
(43) Pub. Date: Sep. 30, 2021

(54) AUGMENTED REALITY FOR A SURGICAL SYSTEM

(71) Applicant: Trumpf Medizin Systeme GmbH & Co. KG., Saalfeld (DE)

(72) Inventor: Tayfur COSKUN, Puchheim (DE)

(21) Appl. No.: 17/188,340

(22) Filed: Mar. 1, 2021

Related U.S. Application Data
(60) Provisional application No. 63/000,529, filed on Mar. 27, 2020.

(51) Int. Cl. A61B 90/30; A61B 90/00

(52) U.S. Cl. CPC A61B 90/30; A61B 2090/365; A61B 90/361

(57) ABSTRACT

A surgical system includes a light head configured to be positioned above a surgical table. A light source is positioned in the light head to direct light onto a patient positioned on the surgical table. An augmented reality projector is coupled to the light head.

### CROSS-REFERENCE TO RELATED APPLICATIONS

This application claims priority under 35 U.S.C. § 119(e) to U.S. Provisional Patent Application Ser. No. 63/000,529, filed Mar. 27, 2020, which is expressly incorporated by reference herein.

### BACKGROUND

The disclosed embodiments include a surgical system, and more particularly, a surgical system that incorporates augmented reality.

Augmented reality utilizes various medical imaging techniques to provide a view of a patient's internal organs. This view of the internal organs may be used to aid in a surgery to limit a number of incisions or a size of incisions by mapping the patient's internal organs during surgery. Generally, the augmented view of the patient's internal organs is provided using virtual reality glasses that are worn by the surgeon during a surgical procedure. Often, the virtual reality glasses inhibit the surgeons ability to properly perform the surgery. That is, the virtual reality glasses may create an obstacle for the surgeon during surgery.

### SUMMARY

The present disclosure includes one or more of the features recited in the appended claims and/or the following features which, alone or in any combination, may comprise patentable subject matter.

According to a first aspect of the disclosed embodiments, a surgical system may include a light head configured to be positioned above a surgical table. A light source may be positioned in the light head to direct light onto a patient positioned on the surgical table. An augmented reality projector may be coupled to the light head and may be configured to project an image of the patient's internal anatomy onto the patient.

In some embodiments of the first aspect, the image of the patient's internal anatomy may be aligned with a corresponding external anatomy of the patient. An anatomical feature of the patient's anatomy may be utilized to align the image of the patient's internal anatomy with the corresponding external anatomy of the patient. The image of the patient's internal anatomy may be aligned with the corresponding external anatomy of the patient in real-time.

It may be desired in the first aspect that a sensor detects movement of the patient. The light head may be moved based on the detected movement of the patient to align the image of the patient's internal anatomy with the corresponding external anatomy of the patient. A control circuitry may receive data from the sensor. The control circuitry may control the movement of the light head to align the image of the patient's internal anatomy with the corresponding external anatomy of the patient. The movement of the light head may cause movement of the light source. The sensor may be positioned near the surgical table and directed toward the surgical table. The sensor may be positioned in the light head.

It may be contemplated in the first aspect that a camera detects movement of the patient. The light head may be moved based on the detected movement of the patient to align the image of the patient's internal anatomy with the corresponding external anatomy of the patient. A control circuitry may receive data from the camera. The control circuitry may control the movement of the light head to align the image of the patient's internal anatomy with the corresponding external anatomy of the patient. The movement of the light head may cause movement of the light source. The camera may be positioned near the surgical table and directed toward the surgical table. The camera may be positioned in the light head.

According to a second aspect of the disclosed embodiments, a surgical system may include a light head configured to be positioned above a surgical table. A light source may be positioned in the light head to direct light onto a patient positioned on the surgical table. An augmented reality projector may be coupled to the light head and may be configured to project an image of the patient's internal anatomy onto the patient. A detection device may detect movement of the patient. The light head may be moved based on the detected movement of the patient to align the image of the patient's internal anatomy with a corresponding external anatomy of the patient.

Optionally in the second aspect, an anatomical feature of the patient's anatomy may be utilized to align the image of the patient's internal anatomy with the corresponding external anatomy of the patient. The image of the patient's internal anatomy may be aligned with the corresponding external anatomy of the patient in real-time.

It may be contemplated in the second aspect that a control circuitry receives data from the detection device. The control circuitry may control the movement of the light head to align the image of the patient's internal anatomy with the corresponding external anatomy of the patient. The movement of the light head may cause movement of the light source. The detection device may be positioned near the surgical table and directed toward the surgical table. The detection device may be positioned in the light head.

According to a third aspect of the disclosed embodiments, a surgical system may include a light head configured to be positioned above a surgical table. A light source may be positioned in the light head to direct light onto a patient positioned on the surgical table. An augmented reality projector may be coupled to the light head and configured to project an image of the patient's internal anatomy onto the patient. An anatomical feature of the patient's anatomy may be utilized to align the image of the patient's internal anatomy with a corresponding external anatomy of the patient in real-time.

In some embodiments of the third aspect, a sensor may detect movement of the patient. The light head may be moved based on the detected movement of the patient to align the image of the patient's internal anatomy with the corresponding external anatomy of the patient. A control circuitry may receive data from the sensor. The control circuitry may control the movement of the light head to align the image of the patient's internal anatomy with the corresponding external anatomy of the patient. The movement of the light head may cause movement of the light source. The sensor may be positioned near the surgical table and directed toward the surgical table. The sensor may be positioned in the light head.

It may be contemplated in the third aspect that a camera detects movement of the patient. The light head may be moved based on the detected movement of the patient to align the image of the patient's internal anatomy with the corresponding external anatomy of the patient. A control circuitry may receive data from the camera. The control circuitry may control the movement of the light head to align the image of the patient's internal anatomy with the corresponding external anatomy of the patient. The movement of the light head may cause movement of the light source. The camera may be positioned near the surgical table and directed toward the surgical table. The camera may be positioned in the light head.

According to a fourth aspect of the disclosed embodiments, A surgical system may include a surgical table. A patient may be configured to be positioned on the surgical table. A light head may be configured to be positioned above a surgical table. A light source may be positioned in the light head to direct light onto the patient positioned on the surgical table. An augmented reality projector may be coupled to the light head and configured to project an image of the patient's internal anatomy onto the patient.

It may be desired in the fourth aspect that the image of the patient's internal anatomy is aligned with a corresponding external anatomy of the patient. An anatomical feature of the patient's anatomy may be utilized to align the image of the patient's internal anatomy with the corresponding external anatomy of the patient. The image of the patient's internal anatomy may be aligned with the corresponding external anatomy of the patient in real-time.

In some embodiments of the fourth aspect, a sensor detects movement of the patient. The light head may be moved based on the detected movement of the patient to align the image of the patient's internal anatomy with the corresponding external anatomy of the patient. A control circuitry may receive data from the sensor. The control circuitry may control the movement of the light head to align the image of the patient's internal anatomy with the corresponding external anatomy of the patient. The movement of the light head may cause movement of the light source. The sensor may be positioned near the surgical table and directed toward the surgical table. The sensor may be positioned in the light head.

Optionally in the fourth aspect, a camera may detect movement of the patient. The light head may be moved based on the detected movement of the patient to align the image of the patient's internal anatomy with the corresponding external anatomy of the patient. A control circuitry may receive data from the camera. The control circuitry may control the movement of the light head to align the image of the patient's internal anatomy with the corresponding external anatomy of the patient. The movement of the light head may cause movement of the light source. The camera may be positioned near the surgical table and direct toward the surgical table. The camera may be positioned in the light head.

According to a fifth aspect of the disclosed embodiments, a method of operating a surgical system may include positioning a patient on a surgical table. The method may also include directing light onto the patient positioned on the surgical table from a light source in a light head. The method may also include projecting an image of the patient's internal anatomy onto the patient from an augmented reality projector coupled to the light head.

In some embodiments of the fifth aspect, the method may also include aligning the image of the patient's internal anatomy with a corresponding external anatomy of the patient. The method may also include utilizing an anatomical feature of the patient's anatomy to align the image of the patient's internal anatomy with the corresponding external anatomy of the patient. The method may also include aligning the image of the patient's internal anatomy with the corresponding external anatomy of the patient in real-time.

It may be desire that the method of the fifth aspect includes detecting movement of the patient with a sensor. The method may also include moving the light head based on the detected movement of the patient to align the image of the patient's internal anatomy with the corresponding external anatomy of the patient. The method may also include transmitting data from the sensor to control circuitry. The method may also include controlling the movement of the light head to align the image of the patient's internal anatomy with the corresponding external anatomy of the patient with the control circuitry. The movement of the light head may cause movement of the light source. The sensor may be positioned near the surgical table and directed toward the surgical table. The sensor may be positioned in the light head.

Optionally, the method of the fifth aspect may also include detecting movement of the patient with a camera. The method may also include moving the light head based on the detected movement of the patient to align the image of the patient's internal anatomy with the corresponding external anatomy of the patient. The method may also include transmitting data from the camera to control circuitry. The method may also include controlling the movement of the light head to align the image of the patient's internal anatomy with the corresponding external anatomy of the patient with the control circuitry. The movement of the light head may cause movement of the light source. The camera may be positioned near the surgical table and directed toward the surgical table. The camera may be positioned in the light head.

### BRIEF DESCRIPTION OF THE DRAWINGS

The detailed description particularly refers to the accompanying figures in which:

FIG. 1 is a side perspective view of a surgical system that incorporates an augmented reality projector;

FIG. 2 is a block diagram of a surgical system having an augmented reality projector; and

FIG. 3 is top plan view of a patient positioned on the surgical table of FIG. 1 showing an augmented view of the patient's internal organs projected onto the patient.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

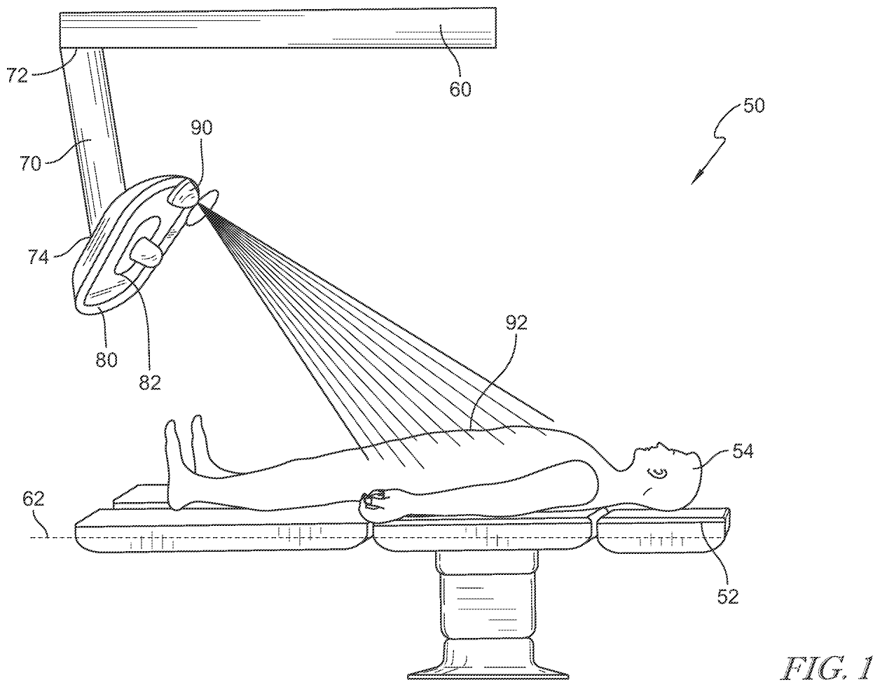

Referring to FIG. 1, a surgical system 50 includes a surgical table 52. A patient 54 is positioned on the surgical table 52 during a surgical procedure. The surgical table 52 may be adjustable in height to move the patient 54 upward or downward relative to a floor. Additionally, the surgical table 52 may be configured to support the patient 54 in any configuration based on movement of various surgical table sections to corresponding positions. For example, the patient 54 may be positioned on the patient's back, on the patient's stomach, or on the patient's side. In some embodiments, the surgical table 52 may be any surgical table known in the art.

A track 60 extends above the surgical table 52. In the illustrative embodiment, the track 60 extends parallel to a longitudinal axis 62 of the surgical table 52. In other embodiments, the track 60 may extend perpendicular to the longitudinal axis 62 or at an angle relative to the longitudinal axis 62. In some embodiments, a plurality of tracks 60 may be provided that extend in different directions.

An arm 70 extends downwardly from the track 60. The arm 70 includes an upper end 72 that is configured to move along the track 60. The arm 70 also includes a lower end 74 opposite the upper end 72. A light head 80 is coupled to the lower end 74 and is configured to move with the arm 70 as the arm 70 is moved along the track 60. A light source 82 is positioned in the light head 80. The light head 80 is angled to direct light from the light source 82 onto the patient 54. The light source 82 may be any conventional light bulb or light source used in a surgical setting.

The light head 80 is configured to rotate side to side and/or up and down relative to the lower end 74 of the arm 70. The light head 80 is moveable to direct light from the light source 82 onto a desired location on the patient 54. Additionally, the arm 70 slides on the track 60 to move the light source 82 relative to the patient 54. Accordingly, the light head 80 is moveable along the track 60 and relative to the lower end 74 of the arm 70 to direct the light from the light source onto the desired area of the patient 54, i.e. the area of the patient being operated on. In other embodiments, multiple articulated arms support the light head 80 relative to a ceiling of an operating room.

An augmented reality projector 90 is coupled to the light head 80 as shown in FIG. 1. In particular, the augmented reality projector 90 is coupled to a peripheral rim of the light head 80 and is aimed in the same general direction that light from the light source 82 is emitted from the light head 80. The augmented reality projector 90 projects an image 92 of the patient's internal organs onto the patient 54. The image 92 may be generated by medical images, i.e. MRI, CT scan, PET scan, x-ray, or any other known medical imaging method. The image 92 corresponds to the area of the patient 54 that is to be operated on. For example, if the patient 54 is having heart surgery, the image 92 is an image of the patient's chest. In some embodiments, the image 92 is updated in real-time.

By positioning the projector 90 on the light head 80, the image 92 is moved along with the light source 82. Accordingly, as the surgeon operates and moves the light source 82 to redirect the light from the light source 82, the image 92 likewise moves. Additionally, by coupling the projector 90 to the light head 80, wiring may be reduced throughout the operating room. That is, wiring to the augmented reality projector 90 is routed through the track 60, the arm 70, and light head 80 so as to be generally hidden from view. Moreover, coupling the projector 90 to the light head 80 eliminates the need to have additional equipment in the operating room.

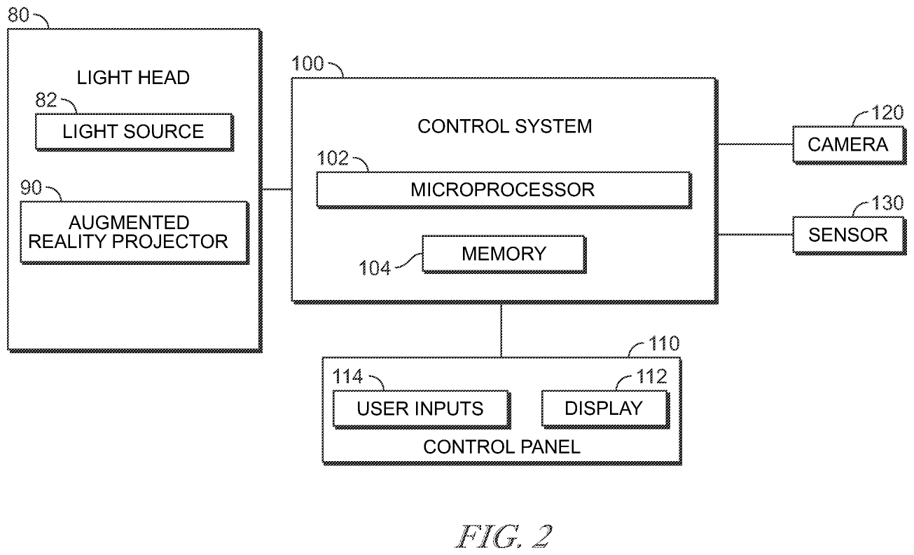

Referring now to FIG. 2, a control system 100 is electronically coupled to the light head 80. The control system 100 includes a processor 102, for example a microprocessor, and a memory 104. The memory 104 retains instructions that are carried out by the processor 102 to operate the light head 80 as described herein. A control panel 110 is electronically coupled to the control system 100. The control panel 110 includes a display 112 and a plurality of user inputs 114. The surgeon or other caregiver may operate the user inputs 114 to control movement of the light head 80. For example, the surgeon may operate the user inputs 114 to rotate the light head 80 relative to the lower end 74 of the arm 70 or to move the arm 70 and light head 80 along the track 60. That is, the surgeon may operate the user inputs 114 to direct the light source 82 and the projector 90 onto a desired area of the patient 54.

A camera 120 is electronically coupled to the control system 100. In some embodiments, the camera 120 is positioned in the operating room near the surgical table 52 and directed toward the surgical table 52. In other embodiments, the camera 120 is coupled to the light head 80. It should be noted that the system 50 may include any number of cameras 120. The camera 120 is configured to monitor a position of the image 92 on the patient 54 to align the image on the patient's external anatomy. If the camera 120 detects that the image 92 is not properly aligned with the patient's external anatomy, the camera 120 sends a signal to the control system 100 to adjust the position of the light head 80 to realign the image 92 with the patient's external anatomy. In some embodiments, the position of the augmented reality projector 90 is movable relative to the light head 80 and the control system 100 commands adjustment of the position of the augmented reality projector 90 relative to the light head 80 while the light head 80 remains stationary based on signals from camera 120. The camera 120 may also detect whether the patient 54 moved during surgery and send a signal to the control system 100 to adjust the light head 80 to realign the image 92 with the patient's external anatomy.

A sensor 130 is also electronically coupled to the control system 100. In some embodiments, the sensor 130 is positioned in the operating room near the surgical table 52 and directed toward the surgical table 52. In other embodiments, the sensor 130 is coupled to the light head 80. It should be noted that the system 50 may include any number of sensors 130. The sensor 130 is configured to monitor a position of the image 92 on the patient 54 to align the image on the patient's external anatomy. If the sensor 130 detects that the image 92 is not properly aligned with the patient's external anatomy, the sensor 130 sends a signal to the control system 100 to adjust the position of the light head 80 and/or the augmented reality projector 90 to realign the image 92 with the patient's external anatomy. The sensor 130 may also detect whether the patient 54 moved during surgery and send a signal to the control system 100 to adjust the position of the light head 80 and/or the augmented reality projector 90 to realign the image 92 with the patient's external anatomy.

It should be noted that the system 50 may be operated with only one of the camera 120 and the sensor 130. In some embodiments, the system 50 may include both the camera 120 and the sensor 130. In yet another embodiments, the system 50 may not include either of the camera 120 or the sensor 130, and the light head 80 may only be operated manually by the surgeon.

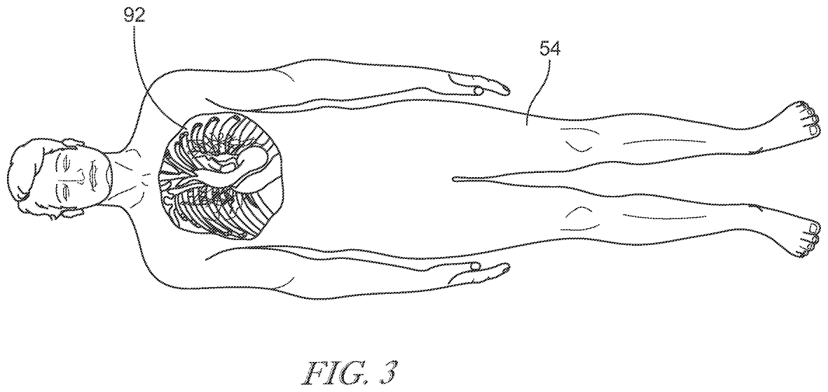

Referring to FIG. 3, the patient 54 is shown positioned on the surgical table 52. The image 92 is illustrated on the patient 54. In the illustrated embodiment, the image 92 is aligned with a corresponding external anatomy of the patient 54. For example, in the illustrated embodiment, the patient's collarbone, which is visible externally may be utilized as a reference point for aligning the image 92 with the patient's external anatomy. In another example, where the surgeon is operating on the patient's hand, the patient's fingertips may be utilized as a reference point for aligning the image 92 with the patient's external anatomy. That is, an anatomical feature of the patient's anatomy is utilized to align the image 92 with the corresponding external anatomy of the patient 54. In some embodiments, using the camera 120 or the sensor 120, the image 92 is aligned with the corresponding external anatomy of the patient 54 in real-time.

Any theory, mechanism of operation, proof, or finding stated herein is meant to further enhance understanding of principles of the present disclosure and is not intended to make the present disclosure in any way dependent upon such theory, mechanism of operation, illustrative embodiment, proof, or finding. It should be understood that while the use of the word preferable, preferably or preferred in the description above indicates that the feature so described can be more desirable, it nonetheless cannot be necessary and embodiments lacking the same can be contemplated as within the scope of the disclosure, that scope being defined by the claims that follow.

In reading the claims it is intended that when words such as “a,” “an,” “at least one,” “at least a portion” are used there is no intention to limit the claim to only one item unless specifically stated to the contrary in the claim. When the language “at least a portion” and/or “a portion” is used the item can include a portion and/or the entire item unless specifically stated to the contrary.

It should be understood that only selected embodiments have been shown and described and that all possible alternatives, modifications, aspects, combinations, principles, variations, and equivalents that come within the spirit of the disclosure as defined herein or by any of the following claims are desired to be protected. While embodiments of the disclosure have been illustrated and described in detail in the drawings and foregoing description, the same are to be considered as illustrative and not intended to be exhaustive or to limit the disclosure to the precise forms disclosed. Additional alternatives, modifications and variations can be apparent to those skilled in the art. Also, while multiple inventive aspects and principles can have been presented, they need not be utilized in combination, and many combinations of aspects and principles are possible in light of the various embodiments provided above.

1. A surgical system comprising:
a light head configured to be positioned above a surgical table,
a light source positioned in the light head to direct light onto a patient positioned on the surgical table, and
an augmented reality projector coupled to the light head and configured to project an image of the patient's internal anatomy onto the patient.

2. The surgical system of claim 1, wherein the image of the patient's internal anatomy is aligned with a corresponding external anatomy of the patient.

3. The surgical system of claim 2, wherein an anatomical feature of the patient's anatomy is utilized to align the image of the patient's internal anatomy with the corresponding external anatomy of the patient.

4. The surgical system of claim 2, wherein the image of the patient's internal anatomy is aligned with the corresponding external anatomy of the patient in real-time.

5. The surgical system of claim 2, further comprising a sensor to detect movement of the patient, wherein the light head is moved based on the detected movement of the patient to align the image of the patient's internal anatomy with the corresponding external anatomy of the patient.

6. The surgical system of claim 5, further comprising a control circuitry to receive data from the sensor, wherein the control circuitry controls the movement of the light head to align the image of the patient's internal anatomy with the corresponding external anatomy of the patient.

7. The surgical system of claim 5, wherein the sensor is positioned in the light head.

8. The surgical system of claim 2, further comprising a camera to detect movement of the patient, wherein the light head is moved based on the detected movement of the patient to align the image of the patient's internal anatomy with the corresponding external anatomy of the patient.

9. The surgical system of claim 8, further comprising a control circuitry to receive data from the camera, wherein the control circuitry controls the movement of the light head to align the image of the patient's internal anatomy with the corresponding external anatomy of the patient.

10. The surgical system of claim 8, wherein the camera is positioned in the light head.

11. A surgical system comprising:
a surgical table, wherein a patient is configured to be positioned on the surgical table,
a light head configured to be positioned above a surgical table,
a light source positioned in the light head to direct light onto the patient positioned on the surgical table, and
an augmented reality projector coupled to the light head and configured to project an image of the patient's internal anatomy onto the patient.

12. The surgical system of claim 11, wherein the image of the patient's internal anatomy is aligned with a corresponding external anatomy of the patient.

13. The surgical system of claim 11, wherein an anatomical feature of the patient's anatomy is utilized to align the image of the patient's internal anatomy with the corresponding external anatomy of the patient.

14. The surgical system of claim 13, wherein the image of the patient's internal anatomy is aligned with the corresponding external anatomy of the patient in real-time.

15. The surgical system of claim 13, further comprising a sensor to detect movement of the patient, wherein the light head is moved based on the detected movement of the patient to align the image of the patient's internal anatomy with the corresponding external anatomy of the patient.

16. The surgical system of claim 15, further comprising a control circuitry to receive data from the sensor, wherein the control circuitry controls the movement of the light head to align the image of the patient's internal anatomy with the corresponding external anatomy of the patient.

17. The surgical system of claim 15, wherein the sensor is positioned in the light head.

18. The surgical system of claim 11, further comprising a camera to detect movement of the patient, wherein the light head is moved based on the detected movement of the patient to align the image of the patient's internal anatomy with the corresponding external anatomy of the patient.

19. The surgical system of claim 18, further comprising a control circuitry to receive data from the camera, wherein the control circuitry controls the movement of the light head to align the image of the patient's internal anatomy with the corresponding external anatomy of the patient.

20. The surgical system of claim 18, wherein the camera is positioned in the light head.
